(19) United States
(12) Patent Application Publication
(10) Pub. No.: US 2024/0197529 A1
(43) Pub. Date: Jun. 20, 2024

(54) GRASPING STRUCTURE FOR OPHTHALMIC SURGERY

(71) Applicant: Alcon Inc., Fribourg (CH)

(72) Inventors: Reto Grüebler, Greifensee (CH); Luca Palmerini, Basel (CH)

(21) Appl. No.: 18/535,676

(22) Filed: Dec. 11, 2023

Related U.S. Application Data
(60) Provisional application No. 63/387,343, filed on Dec. 14, 2022.

(51) Int. Cl. A61F 9/007

(52) U.S. Cl. CPC A61F 9/00754

(57) ABSTRACT

Particular embodiments disclosed herein provide a surgical instrument for grasping a retinal membrane. An ophthalmic surgical instrument for peeling a retinal membrane includes a handle having an actuator mounted on the handle. An outer tube has a proximal end mounted to the handle. First and second arms extend outwardly from the outer tube and each have pulling surfaces thereon. The pulling surfaces are configured such that when the outer tube is in first position, the pulling surfaces flare outwardly from one another and may both be placed simultaneously in contact with the retinal membrane. The arms may be made of a flexible material to conform to the retinal membrane. When the outer tube is in a second position, the pulling surfaces are facing one another and pressed against one another. The pulling surfaces may include barbs that point toward the outer tube when the pulling surfaces are drawn together.

### BACKGROUND

The internal limiting membrane (ILM) is a thin transparent membrane positioned between the vitreous and the retina of the eye. The ILM plays a role during the formation of the eye but is not required for the proper function of an adult eye. The ILM may pull at the retina and cause conditions such as macular holes, macular pucker, vitreo-macular traction syndrome, diabetic macular edema, and cystoid macular edema secondary to inflammation or venous occlusive diseases and other conditions. An epiretinal membrane (ERM) is a membrane that may form over the retina in response to damage to the retina, such as due to posterior vitreous detachment.

The ILM or ERM may need to be peeled away from the retina to prevent damage to the retina. Peeling of the ILM or ERM may also be required in preparation for surgical procedures performed on the retina. To peel the ILM or ERM, a surgical instrument is inserted through a cannula (e.g., trocar cannula) within the patient's eye globe. Forceps or a specialized scraper are extended from the instrument and used to raise a flap in the ILM or ERM. The flap is then grasped by the forceps and the ILM or ERM is peeled away from the retina using a circular motion. Excess force on the forceps may result in piercing of the retina.

It would, therefore, be an advancement in the art to reduce the risk of retinal damage resulting from ILM peeling.

### BRIEF SUMMARY

The present disclosure relates generally to a structure for grasping the internal limiting membrane (ILM) or epiretinal membrane (ERM) during ophthalmic surgery.

An ophthalmic surgical instrument for peeling a retinal membrane includes a handle having an actuator mounted on the handle. An outer tube has a proximal end mounted to the handle. A first arm extends outwardly from a distal end of the outer tube and includes a first distal portion having a first pulling surface formed thereon. A second arm extends outwardly from the distal end of the outer tube and includes a second distal portion having a second pulling surface formed thereon.

The first pulling surface and second pulling surface are configured such that (a) when the outer tube is in first position relative to the first arm and the second arm, the first pulling surface and the second pulling surface flare outwardly from one another and may both be placed simultaneously in contact with the retinal membrane and (b) when the outer tube is in a second position relative to the first arm and the second arm the first pulling surface and the second pulling surface are facing one another and pressed against one another.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A is an isometric view of a surgical instrument having a grasping structure including pulling and gripping surfaces, in accordance with certain embodiments.

FIG. 1B is an isometric view of another surgical instrument having a grasping structure including pulling and gripping surfaces, in accordance with certain embodiments.

FIG. 2A is cross-sectional view of the grasping structure in a retracted configuration, in accordance with certain embodiments.

FIG. 2B is a front view of the grasping structure in a retracted configuration, in accordance with certain embodiments.

FIGS. 3A to 3B are side views illustrating a method of grasping a membrane using the grasping structure, in accordance with certain embodiments.

FIG. 4 is an isometric view showing an ILM being peeled using the grasping structure, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide a surgical instrument including flexible loops for peeling a membrane from a patient's retina. Note that, herein, a distal end of a component refers to the end that is closer to a patient's body while the proximal end of the component refers to the end that is facing away from the patient's body or in proximity to, for example, the handle of the surgical instrument.

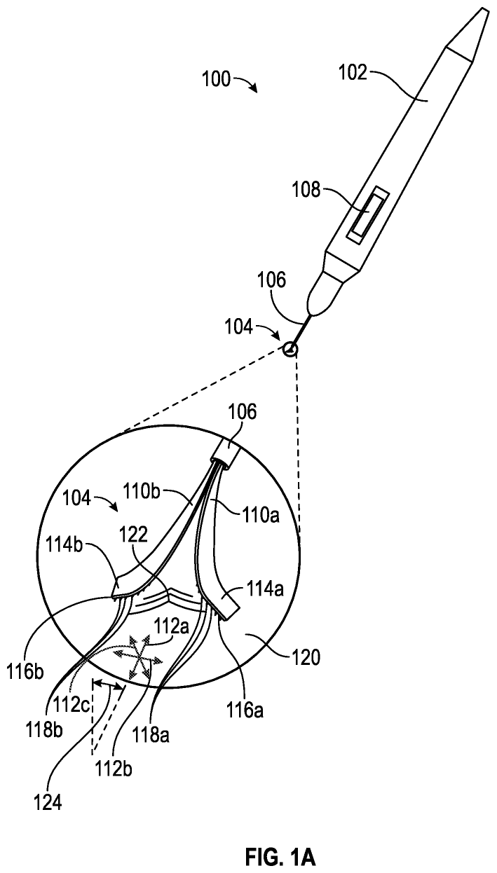

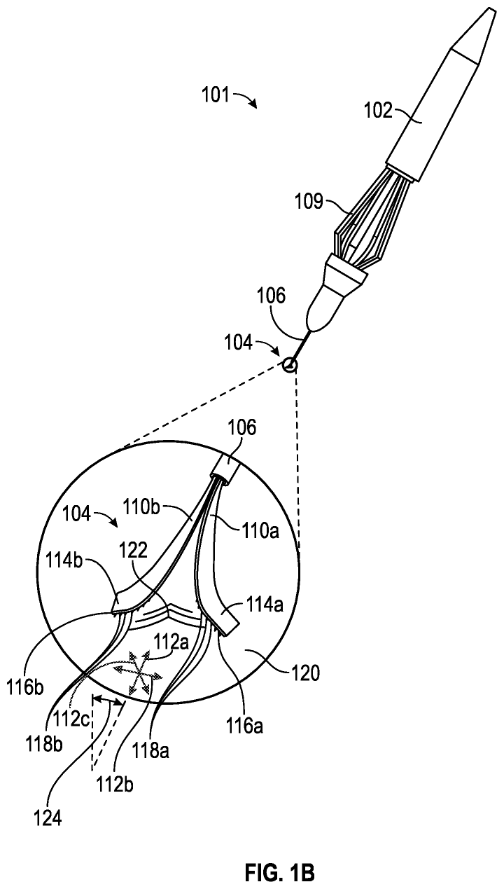

FIG. 1A illustrates a surgical instrument 100, in accordance with certain embodiments, including a handle 102 that is sized and contoured to be grasped by a hand of a surgeon performing an ophthalmic surgical procedure such as peeling of a membrane from a retina of a patient's eye, such as an ILM or ERM. A grasping structure 104 is extendable from a distal end of an outer tube 106 connected to the handle 102. This proximal end of the outer tube 106 is connected to the handle 102. The handle 102 may have a manual control structure mounted thereto. In the embodiment of FIG. 1, the manual control structure includes a slider 108. However, the manual control structure may also be implemented, such as a deformable basket (described below with reference to FIG. 1B), a button, or any other manual control structure known in the art of ophthalmic surgical instruments.

The outer tube 106 is coupled to the slider 108 and moves outwardly from the handle 102 responsive to movement of the slider 108 toward the distal end of the outer tube 106 and moves inwardly into the handle responsive to movement of the slider 108 away from the distal end of the outer tube 106. In other embodiments, the slider 108 is coupled to the grasping structure 104 such that the grasping structure is moved relative to the outer tube 106 responsive to movement of the slider 108.

In FIG. 1A, the grasping structure 104 is embodied as arms 110a, 110b that pass through the outer tube 106 and are fixed relative to the handle 102 when the outer tube is actuated by the slider 108. As such, at least part of the arms 110a, 110b are housed within the outer tube 106. In other implementations, the outer tube 106 is fixed relative to the handle 102 and the arms 110a, 110b are coupled to the slider 108. The outer tube 106 defines a longitudinal direction 112a that is parallel to and collinear with an axis of symmetry of the outer tube 106. When extended from the outer tube 106, the arms 110a, 110b are offset from one another along a transverse direction 112b that is perpendicular to the longitudinal direction 112a. A vertical direction 112c may be defined as perpendicular to the longitudinal direction 112a and the transverse direction 112b.

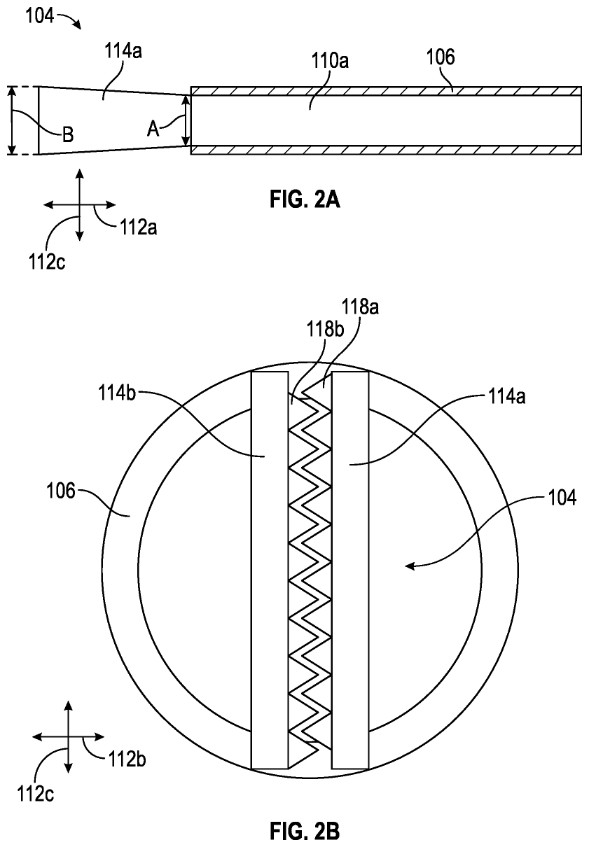

A distal portion of each arm 110a, 110b may include flared portion 114a, 114b. The flared portions 114a, 114b each define pulling surfaces 116a, 116b. When extended from the outer tube, the flared portions 114a, 114b recoil outwardly from the longitudinal direction 112a. The flared portions may also be flared in the sense that the heights of the flared portions 114a, 114b in the vertical direction 112c increase with distance from the distal end of the outer tube 106 along at least a portion of the arm 110a, 110b. For example, each arm 110a, 110b may flare from a height in the vertical direction 112c of A to a height of B at a widest point on the flared portion 114a, 114b (an illustration of A and B is shown in FIG. 2A). The ratio of B to A may be greater than 1.5, greater than 2, greater than 4, or greater than 8. The heights may increase monotonically up to the distal end of the flared portions 114a, 114b or the flared portions 114a, 114b may include a distal portion of constant height.

The pulling surfaces 116a, 116b may have barbs 118a, 118b formed thereon. The barbs 118a, 118b facilitate drawing a membrane 120 between the flared portions 114a, 114b to form a flap 122 and grasp the flap 122. When the pulling surfaces 116a, 116b are pressed flat on the membrane 120, the barbs 118a, 118b may point inwardly such that as the flared portions 114a, 114b are drawn together, the barbs tend to pull on the membrane 120 in order to raise the flap 122. The barbs 118a, 118b may be embodied as an array of barbs 118a, 118b resembling the barbed scales of sharkskin.

The barbs 118a, 118b may be oriented such that sliding of the pulling surfaces 116a, 118b outwardly from the longitudinal direction 112a along the membrane 120 is resisted much less than sliding of the pulling surfaces 116a, 116b along the membrane 120 inwardly toward the longitudinal direction 112a. In this manner, the pulling surfaces 116a, 116b may be pressed against the membrane 120 causing the pulling surfaces 116a, 116b to spread and press flat against the membrane 120. When the pulling surfaces 116a, 116b are pulled together in response to extending the outer tube 106 around the arms 110a, 110b, the barbs 118a, 118b will grab the membrane 120 and raise the flap 122.

The pulling surfaces 116a, 116b may have various relative orientations absent a deforming force when extended from the outer tube 106. The pulling surfaces 116a, 116b may each flare outwardly at an angle of at least 45 degrees, at least 60 degrees, at least 90 degrees, or at least 90 degrees relative to the longitudinal direction 112a in a plane parallel to the longitudinal direction 112a and the transverse direction 112b. In use, the grasping structure 104 may be pressed against a membrane 120 and the flared portions 114a, 114b are pushed outwardly until the pulling surfaces 116a, 116b are resting on the membrane 120.

The pulling surfaces 116a, 116b may be planar oriented substantially parallel (e.g., within 10 degrees) to one another and be substantially co-planar (e.g., within 0.1 mm (millimeters)) with one another when the arms 110a, 110b are extended and absent a deforming force. The pulling surfaces 116a, 116b may conform to a circle (e.g., be within 0.1 mm of at all points), the circle being in a plane parallel to the longitudinal direction 112a and transverse direction 112b. The radius of the circle may correspond to the curvature of a patient's retina, e.g., be equal to or slightly (e.g., 1 mm) smaller than the curvature of a patient's retina. The pulling surfaces 116a, 116b may likewise conform to a sphere having a radius as defined above for the circle. The flared portions 114a, 114b may be sufficiently flexible such that even when the pulling surfaces 116a, 116b are planar, the flared portions 114a, 114b may deform to conform to the curved retina of the patient.

The longitudinal direction 112a may be substantially (e.g., within 10 degrees of) normal to the pulling surfaces 116a, 116b. In other implementations, the longitudinal direction 112a may define an angle 124 with respect to the pulling surfaces 116a, 116b. The outer tube 106 is typically inserted through a trocar cannula in the sclera of the eye to one side of the pupil. The membrane 120 to be peeled may be directly facing the pupil such that outer tube 106 may be at a slight angle relative to the membrane 120. The pulling surfaces 116a, 116b may be angled with respect to the longitudinal direction 112a to account for this angle. For example, as shown in FIG. 1, the pulling surfaces may define the angle 124 with respect to a plane that is parallel to the transverse direction 112b and the vertical direction 112c. In other implementations, the flared portions 114a, 114b may be sufficiently flexible to flex to conform to the membrane 120 notwithstanding a non-zero angle of the outer tube 106 relative to the normal vector of the membrane 120.

The grasping structure 104 may be made of a highly flexible material, such as nitinol (a nickel titanium alloy), spring steel, polymeric materials, or other material. The high flexibility enables grasping structure 104 to deform elastically when withdrawn into the outer tube 106 and to conform to the membrane 120. When extended from the outer tube 106, flared portions 114a, 114b may expand to a width in the transverse direction 112b that is many times the inner diameter of the outer tube 106 and possibly many times the outer diameter of the outer tube, such as 1.5, two, four, or eight times.

FIG. 1B illustrates another surgical instrument 101, in accordance with certain embodiments. As shown, surgical instrument 101 is substantially similar to surgical instrument 100, but for the manual control structure of handle 102. In the embodiments of FIG. 1B, the manual control structure comprises a deformable basket 109. In such embodiments, the outer tube 106 is coupled to the deformable basket 109 and moves outwardly from the handle 102 responsive to compression of the deformable basket 109, and moves inwardly into the handle 102 responsive to decompression of the deformable basket 109. In other embodiments, the deformable basket 109 is coupled to the grasping structure 104 such that the grasping structure is moved relative to the outer tube 106 responsive to compression and decompression of the deformable basket 109.

Referring to FIGS. 2A and 2B, when inserting the grasping structure 104 through a trocar cannula and withdrawing the grasping structure 104 through the trocar cannula, the outer tube 106 is extended over the arms 110a, 110b. The flared portions 114a, 114b may fit within the outer tube 106 or deform elastically to fit within the outer tube 106. Alternatively, the flared portions 114a, 114b may remain outside the outer tube 106, as shown in FIG. 2B, but be pressed together and fit within the outer diameter of the outer tube 106 such that the flared portions 114a, 114b may pass through the trocar cannula without interference.

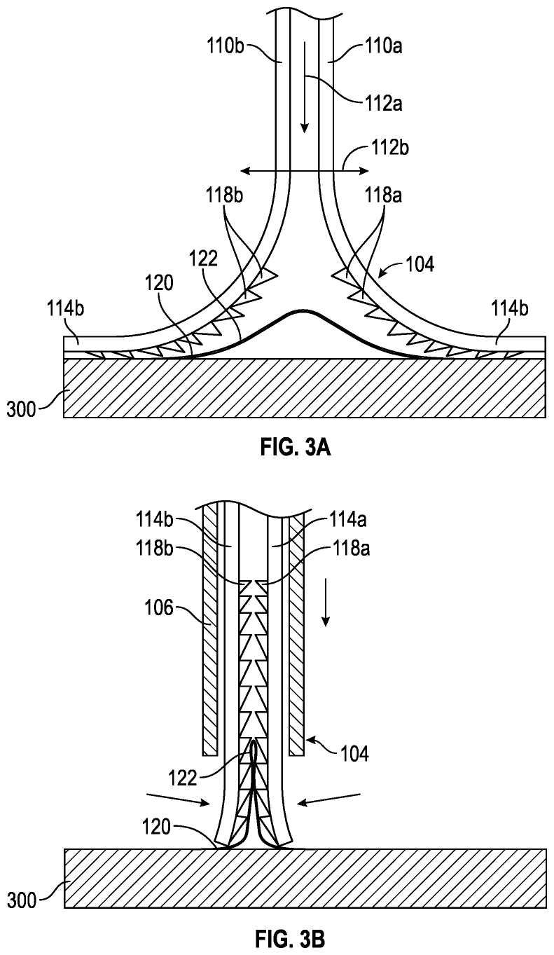

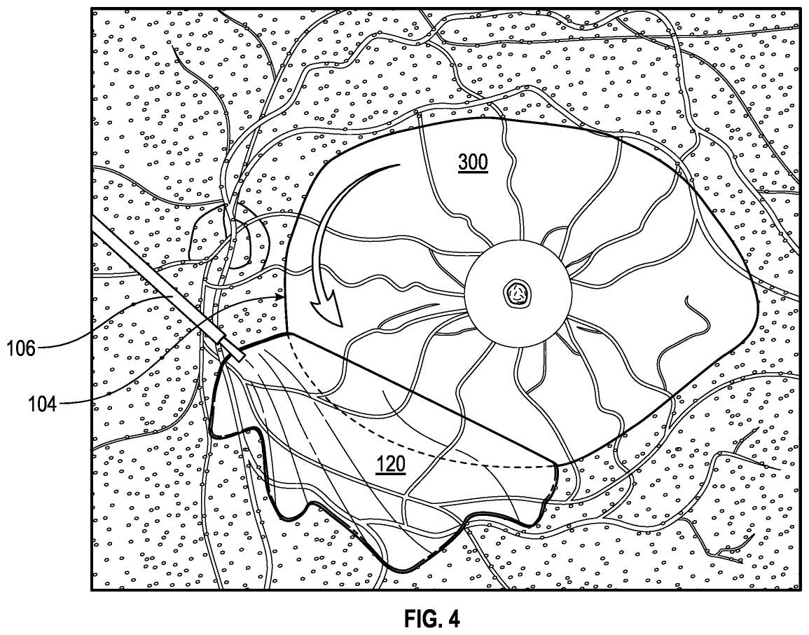

FIGS. 3A and 3B further illustrate the process of peeling the membrane 120 using the grasping structure 104. Referring specifically to FIG. 3A, the outer tube 106 is withdrawn along the longitudinal direction 112a (upwardly in FIG. 3A) to a first position in which the arms 110a, 110b extend outwardly from the outer tube 106 and the flared portions 114a, 114b recoil such that the flared portions 114a, 114b flare outwardly from the outer tube 106. The pulling surfaces 116a, 116b are placed on the membrane 120, which may cause the pulling surfaces 116a, 116b to spread further apart. The barbs 118a, 118b partially penetrate the membrane 120 without penetrating the underlying retina 300. The extent of the barbs 118a, 118b from the pulling surfaces 116a, 116b is preferably less than the thickness of the membrane 120, such as between 0.5 and 0.75 times the thickness of the membrane 120, which may have a thickness of between about 2 μm (micrometers) to about 10 μm. For example, in certain embodiments, the extent of the barbs 118a, 118b from the pulling surfaces 116a, 116b is between about 1 μm and about 7.5 μm, between about 2 μm and about 7 μm, 3 μm and about 6 μm, or between about 4 μm and about 5 μm.

The outer tube 106 is then extended to a second position shown in FIG. 3B in which the arms 110a, 110b are drawn into the tube 106 and the pulling surfaces 116a, 116b are drawn together as shown in FIG. 3B. As the pulling surfaces 116a, 116b are drawn together, the flap 122 of the membrane 120 is raised and is eventually gripped firmly between the pulling surfaces 116a, 116b. The orientation of the barbs 118a, 118b further resists removal of the flap 122 from between the pulling surfaces 116a, 116b. As shown in FIG. 3B, the barbs 118a, 118b point upwardly toward the outer tube 106. As shown in FIG. 4, the surgeon may pull on the flap 122 to induce a tear and move the grasping structure 104 in a circular motion to peel a portion of the membrane 120 away from the retina 300.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. An ophthalmic surgical instrument for peeling a retinal membrane, comprising:
a handle;
an outer tube having a proximal end mounted to the handle and a distal end;
a first arm at least partly housed within the outer tube and including a first distal portion having a first pulling surface formed thereon; and
a second arm at least partly housed within the outer tube and including a second distal portion having a second pulling surface;
an actuator mounted to the handle and configured to change relative positions of the outer tube and the first arm and the second arm;
wherein the first pulling surface and second pulling surface are configured such that (a) when the outer tube is in a first position relative to the first arm and the second arm, the first pulling surface and the second pulling surface are configured to flare outwardly from one another and can both be placed simultaneously in contact with the retinal membrane and (b) when the outer tube is in a second position relative to the first arm and the second arm, the first pulling surface and the second pulling surface are facing one another and pressed against one another.

2. The ophthalmic surgical instrument of claim 1, wherein the actuator is configured to move the outer tube from the first position to the second position responsive to movement of the actuator in a first direction.

3. The ophthalmic surgical instrument of claim 2, wherein the actuator is configured to move the outer tube from the second position to the first position responsive to movement of the actuator in a second direction opposite the first direction.

4. The ophthalmic surgical instrument of claim 1, wherein the first pulling surface and the second pulling surface are configured to flare outwardly from an outer diameter of the outer tube when extended from the distal end of the outer tube.

5. The ophthalmic surgical instrument of claim 1, wherein the first pulling surface and the second pulling surface are configured to flare outwardly to at least twice an outer diameter of the outer tube when extended from the distal end of the outer tube.

6. The ophthalmic surgical instrument of claim 1, wherein the first pulling surface and the second pulling surface are configured to recoil outwardly to an angle of at least 45 degrees relative to an axis of symmetry of the outer tube.

7. The ophthalmic surgical instrument of claim 6, wherein the first arm and the second arm each comprise nitinol.

8. The ophthalmic surgical instrument of claim 1, wherein a first pulling surface includes first barbs configured to grip the retinal membrane and the second pulling surface includes second barbs configured to grip the retinal membrane.

9. The ophthalmic surgical instrument of claim 8, wherein the first barbs and the second barbs have lengths between 1 μm (micrometers) and 7.5 μm.

10. The ophthalmic surgical instrument of claim 8, wherein the first barbs and the second barbs both point toward the outer tube when the outer tube is in the second position.

11. A method for peeling a retinal membrane from a retina of a patient's eye, the method comprising:
inserting a distal end of an outer tube through a trocar cannula in the patient's eye;
extending a first arm and a second arm from the distal end of the outer tube such that a first pulling surface on the first arm and a second pulling surface on the second arm flare outwardly from one another;
engaging the retinal membrane with the first pulling surface on the first arm and the second pulling surface; and
bringing the first arm and the second arm together such that the first pulling surface is facing the second pulling surface with a flap of the retinal membrane grasped between the first pulling surface and the second pulling surface.

12. The method of claim 11, further comprising pulling on the flap effective to peel a portion of the retinal membrane from the retina.

13. The method of claim 11, wherein:
the outer tube is mounted to a handle having an actuator mounted thereto and coupled to the outer tube;
extending the first arm and the second arm from the distal end of the outer tube comprises moving the actuator in a first direction to withdraw the outer tube from over the first arm and the second arm; and
bringing the first arm and the second arm together comprises moving the actuator in a second direction opposite the first direction to extend the outer tube over the first arm and the second arm.

14. The method of claim 11, wherein the first pulling surface and second pulling surface extend outwardly from an outer diameter of the outer tube when extended from the distal end of the outer tube.

15. The method of claim 11, wherein the first pulling surface and second pulling surface extend outwardly from an outer diameter of the outer tube to at least twice an outer diameter of the outer tube when extended from the distal end of the outer tube.
